(19) United States
(12) Patent Application Publication
(10) Pub. No.: US 2011/0313516 A1
(43) Pub. Date: Dec. 22, 2011

(54) PERCUTANEOUS MAGNETIC CATHETER

(75) Inventors: Ninh H. Dang, Trabuco Canyon, CA (US); Samuel M. Shaolian, Newport Beach, CA (US); Ross Tsukashima, San Diego, CA (US); Daniel C. Anderson, Pomona, CA (US)

(73) Assignee: MICARDIA CORPORATION, Irvine, CA (US)

(21) Appl. No.: 13/148,743

(22) PCT Filed: Feb. 12, 2010
(86) PCT No.: PCT/US2010/024102

§ 371 Date: Aug. 10, 2011

Related U.S. Application Data
(60) Provisional application No. 61/152,135, filed on Feb. 12, 2009.

(51) Int. Cl. A61F 2/24

(52) U.S. Cl. 623/2.11

(57) ABSTRACT

A percutaneous magnetic catheter may be used to position one or more magnetic components near a magnetically driven prosthesis in a patient's heart in order to adjust the magnetically driven prosthesis. In certain embodiments, the one or more magnetic components are rotatable. Rotation of the one or more magnetic components cause variations in a magnetic field. Variations in the magnetic field may be utilized to adjust the size or shape of a magnetically driven prosthesis. In this way, a magnetically driven prosthesis may be adjusted after a patient has recovered from a surgery in which the magnetically driven prosthesis was implanted. Further, a magnetically driven prosthesis may be adjusted based upon progression of a heart condition.

### TECHNICAL FIELD

Disclosed herein are systems and methods related to a percutaneous magnetic catheter. The percutaneous magnetic catheter may be used in connection with an adjustable magnetically driven prosthesis.

### BRIEF DESCRIPTION OF THE DRAWINGS

FIG. 1A illustrates a block diagram of one embodiment of a percutaneous magnetic catheter.

FIG. 1B schematically illustrates a magnetic component that is usable in a magnetically driven prosthesis and/or in a percutaneous magnetic catheter according to one embodiment.

FIG. 1C is a cross-sectional view of the magnetic component shown in FIG. 1B according to one embodiment.

FIGS. 1D and 1E schematically illustrate an end view of the magnet component shown in FIG. 1B in proximity to a magnetically driven component within an adjustable magnetically driven prosthesis according to one embodiment.

FIG. 2 illustrates a partial cross-sectional view of one embodiment of a percutaneous magnetic catheter.

FIG. 3 illustrates a percutaneous magnetic catheter positioned in the right atrium of a heart according to one embodiment.

FIG. 4 illustrates a percutaneous magnetic catheter that is deflectable in multiple planes according to one embodiment.

FIG. 5 illustrates one embodiment of a percutaneous magnetic catheter including a protective jacket that surrounds a magnetic component.

FIG. 6 illustrates one embodiment of a percutaneous magnetic catheter that is manipulable in three dimensions.

FIGS. 7A and 7B illustrate various attributes of a flexible driveshaft that may be utilized in connection with a percutaneous magnetic catheter according to certain embodiments.

FIG. 7C illustrates a partial cross-sectional view of one embodiment of a flexible driveshaft.

FIG. 8A is a partially transparent top view of an adjustable magnetically driven prosthesis in an anterior/posterior extended or plus position according to one embodiment.

FIG. 8B is a partially transparent top view of the adjustable magnetically driven prosthesis of FIG. 8A in an anterior/posterior retracted or minus position.

FIG. 8C schematically illustrates a side view of the adjustable magnetically driven prosthesis of FIG. 8A.

FIG. 8D is a partially transparent perspective view of the adjustable magnetically driven prosthesis of FIG. 8A.

FIG. 8E is another partially transparent top view of the adjustable magnetically driven prosthesis of FIG. 8A.

FIG. 9 illustrates a transeptal approach for adjusting an adjustable magnetically driven prosthesis using a percutaneous magnetic catheter according to one embodiment.

FIG. 10 illustrates one embodiment of a percutaneous magnetic catheter that includes two magnetic components.

FIGS. 11A, 11B, 11C, and 11D schematically illustrate end views of a plurality of magnetic components according to one embodiment.

FIG. 12A illustrates an embodiment of a percutaneous magnetic catheter that includes two magnetic components positioned in the left atrium of a heart and two magnetic components positioned in the left ventricle of the heart.

FIG. 12B schematically illustrates a cross-sectional view of the embodiment illustrated in FIG. 12A.

FIG. 13 schematically illustrates a cross-sectional view of an embodiment of a percutaneous magnetic catheter that includes three magnetic components positioned in the left atrium of a heart and three magnetic components positioned in the left ventricle of the heart.

FIG. 14 illustrates a configuration of a magnetic catheter drive system according to one embodiment.

### DETAILED DESCRIPTION OF PREFERRED EMBODIMENTS

Mitral valve defects, such as regurgitation, may be caused by a relaxation of the tissue surrounding the mitral valve. This relaxation may cause the mitral opening to enlarge, which prevents the valve leaflets from sealing properly. This heart condition may be treated by sewing an adjustable magnetically driven prosthesis around the valve. Synching the tissue to the adjustable magnetically driven prosthesis may restore the valve opening to its approximate original size and operating efficiency.

The proper degree of synching, however, may be difficult to determine during open heart surgery. This is due, at least in part, to the fact that the patient is under general anesthesia, in a prone position, with an open chest, and a large incision in the heart. These factors affect the normal operation of the mitral valve. Even if the synching is done well, the tissue may continue to relax over the patient's lifetime such that the heart condition returns.

An adjustable magnetically driven prosthesis may allow for the proper degree of synching both following an open heart surgery and over the patient's lifetime. A percutaneous magnetic catheter according to the present disclosure may be used to position magnetic drive components near the adjustable magnetically driven prosthesis in a patient's heart. The magnetic drive components may be selectively activated so as to cause the adjustable magnetically driven prosthesis to change its size or shape, and thus adjust the synching of the adjustable magnetically driven prosthesis.

Although the present disclosure is presented in terms of systems and methods for adjusting a magnetically driven prosthesis associated with a mitral valve, it is contemplated that the systems and methods disclosed herein may also be employed in the treatment of other conditions. Such conditions include, but are not limited to defects of the aortic valve, tricuspid valve, and pulmonary valve.

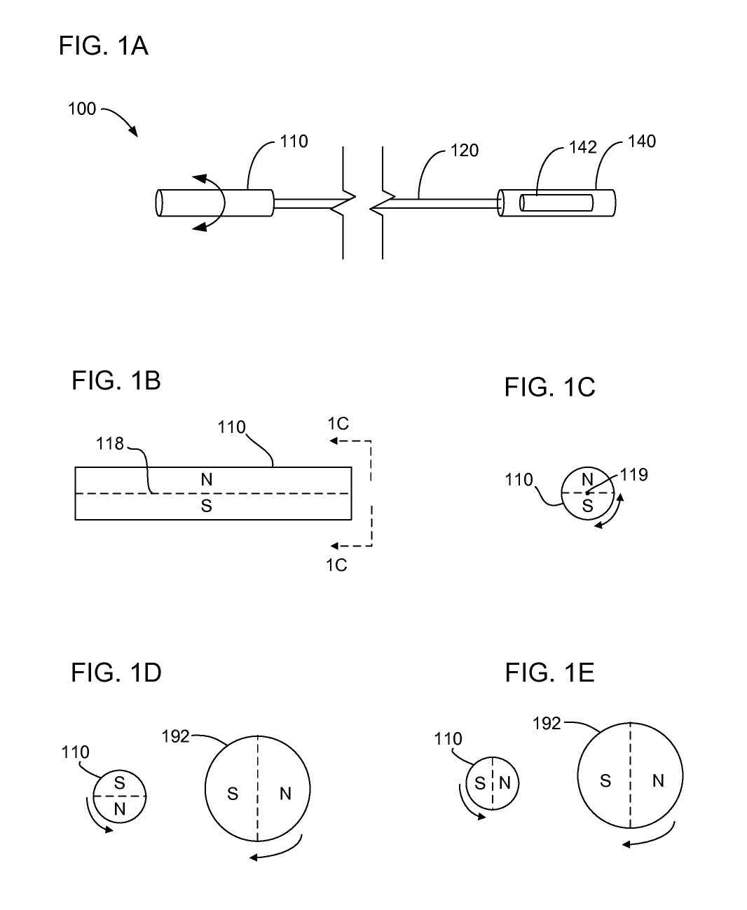

FIG. 1A illustrates a block diagram of one embodiment of a magnetic component 110 connected to a drive component 140 with a drive motor 142. The drive motor 142 causes the magnetic component 110 to rotate in either a clockwise or counterclockwise direction. A flexible driveshaft 120 may connect the drive component 140 with the magnetic component 110.

FIG. 1B schematically illustrates the magnetic component 110 according to one embodiment. FIG. 1C is a cross-sectional view of the magnetic component shown in FIG. 1B. As discussed below, a similar magnetic element may be used in an adjustable magnetically driven prosthesis. In this example, the magnetic component 110 is cylindrical and has magnetic poles (e.g., north “N” and south “S”) divided along a plane 118 that runs the length of the cylinder. The magnetic component 110 rotates around an axis 119.

FIGS. 1D and 1E schematically illustrate an end view of the magnetic component 110 and a magnetically driven component 192 (e.g., located in an adjustable magnetically driven prosthesis such as an annuloplasty ring), which is placed in parallel with the magnetic component 110. Although the magnetic component 110 of the catheter 100 is shown as being smaller than the magnetically driven component 192, in other embodiments the magnetic components 110, 192 are substantially the same size. In yet other embodiments, the magnetic component 110 may be larger than the magnetically driven magnetic component 192. For illustrative purposes, FIG. 1D illustrates the magnetic component 110 and the magnetically driven component 192 aligned for maximum (peak) torque transmission. FIG. 1E illustrates the south pole of the magnetic component 192 aligned with the north pole of the magnetically driven component 110. In this alignment, no torque will be transmitted from the magnetic component 110 to the magnetically driven component 192.

Regardless of a current or initial alignment of the magnetic component 110 and the magnetically driven component 192, the magnetic fields of the components 110, 192 interact such that rotation of the magnetic component 110 causes rotation of the magnetically driven component 192. For example, rotating the magnetic component 110 in a clockwise direction around its cylindrical axis 119 causes the driven magnetic component 192 to rotate in a counterclockwise direction. Similarly, rotating the magnetic component 110 in a counterclockwise direction around its cylindrical axis 119 causes the driven magnetic component 192 to rotate in a clockwise direction.

If the magnetically driven component 192 is suitably coupled to an adjustable magnetically driven prosthesis, the magnetic component 110 may be utilized to adjust the magnetically driven prosthesis. Further, one full rotation of the magnetic component 110 provides accurate one-to-one rotation of the driven magnetic component 192, assuming sufficient magnetic coupling. If the relationship between the number of rotations of the magnetic component 110 and the original size of the adjustable magnetically driven prosthesis is known, the size of the prosthesis may be determined directly after an adjustment from the number of revolutions. Imaging techniques (e.g., using X-ray or ultrasound) may also be used to determine the prosthesis size after it is implanted in the patient.

In various embodiments, the rotational torque on the magnetically driven component 192 may be increased by increasing the strength of the magnetic field of the magnetic component 110 and/or by utilizing a plurality of magnetic components. In embodiments including a plurality of magnetic components, the plurality of magnetic components may be oriented and rotated relative to each other such that their magnetic fields add together to form a combined magnetic field that provides increased rotational torque to the magnetically driven component 192. A computer controlled motor (not shown) may be utilized to synchronously rotate the plurality of magnetic components in order to optimize the strength of the combined magnetic field.

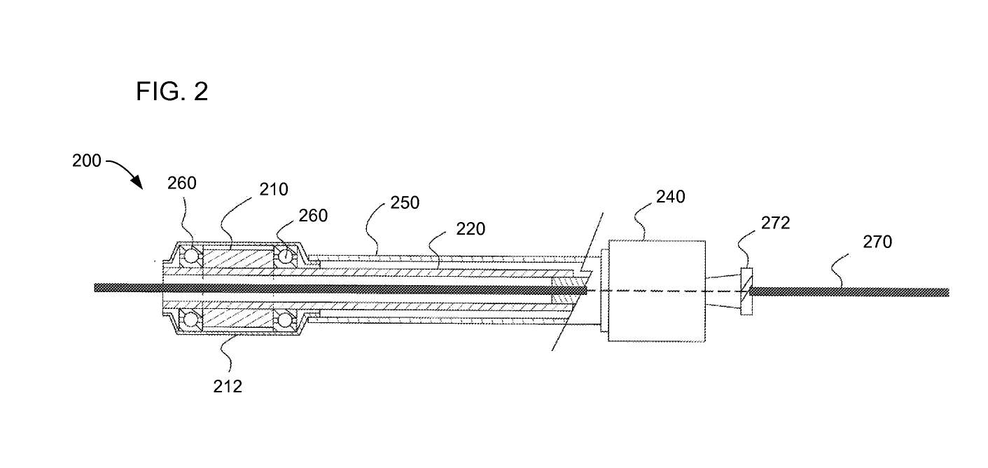

FIG. 2 illustrates a partial cross-sectional view of one embodiment of a percutaneous magnetic catheter 200, including a guide wire 270. The guide wire 270 may be braced against chamber walls in a patient's heart to stabilize a magnetic component 210 as it is rotated during use. The guide wire 270 may be manipulated and utilized to hold a sealed housing 212 in place during adjustment of an adjustable magnetically driven prosthesis. In various embodiments, the magnetic catheter 200 may be locked onto the guide wire 270 using a Tuohy Borst adapter (not shown) on a luer connector 272. The guide wire 270 may pass through ports or lumens in the sealed housing 212.

The percutaneous magnetic catheter 200 may include a drive component 240, which is connected to the magnetic component 210 by way of a flexible driveshaft 220. In various embodiments, the flexible driveshaft 220 may be lined with a material, such as polytetrafluoroethylene (PTFE), in order to minimize lock up and whip effects. The drive component 240 may be configured to provide variable speed, torque, and rotation (e.g., clockwise or counterclockwise rotation).

The sealed housing 212 may also comprise a thin needle or ball bearing 260. The bearing 260 may allow for lower rotational force provided by the flexible driveshaft 220 and may also prevent the magnetic component 210 from scraping against the sealed housing 212 while in operation.

The flexible driveshaft 220 may transfer rotational motion from the drive component 240 to the magnetic component 210 located within the sealed housing 212. The drive component 240 may be configured to rotate the magnetic component 210 in either a clockwise or counterclockwise direction. The flexible driveshaft 220 may pass through a central lumen of a catheter 250.

In certain embodiments, the magnetic component 210 may be positioned in a patient's right atrium, and may be used for activating an adjustable magnetically driven prosthesis (not shown) that was previously implanted. The magnetic component 210 may be positioned close to a patient's septal wall, near the fossa ovalis. In this position, the magnetic component 210 may be sufficiently proximate to the adjustable magnetically driven prosthesis so as to magnetically couple to a magnetically driven component (not shown) in the magnetically driven prosthesis. In various embodiments, adjustment may include altering the prosthesis's commissure-to-commissure distance or the prosthesis's septal-lateral dimensions.

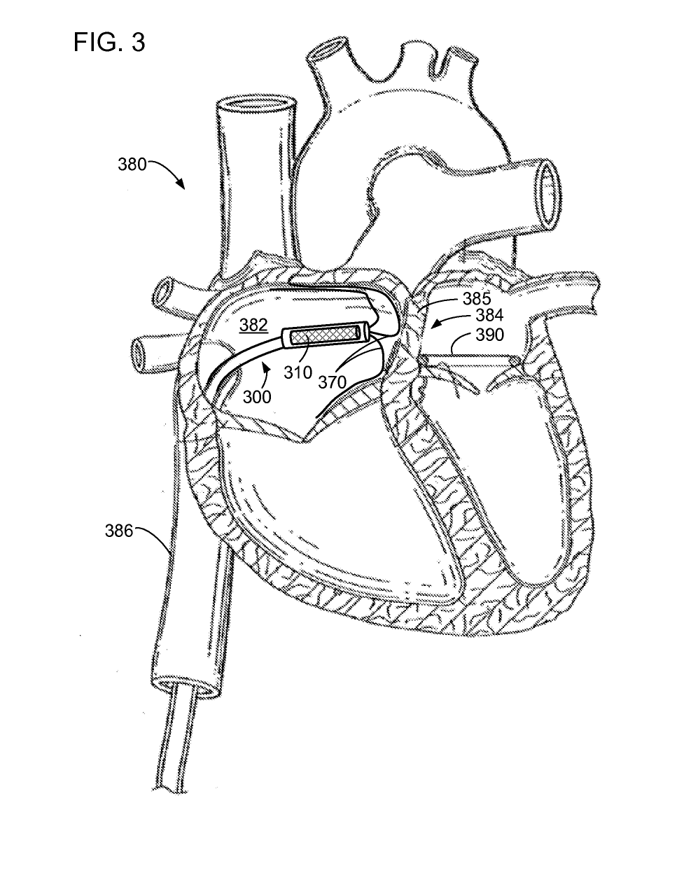

FIG. 3 illustrates a view of one embodiment of a percutaneous magnetic catheter 300 positioned in the right atrium 382 of a heart 380. In certain embodiments, including the illustrated embodiment, a magnetic component 310 may be positioned near the fossa ovalis 384. In this position, the magnetic component 310 is positioned close to a magnetically driven prosthesis 390 (e.g., annuloplasty ring) implanted on the mitral valve without traversing the septal wall 385. As illustrated in FIG. 3, guide wires 370 may be positioned against the walls of the right atrium 382 in order to maintain the position of the percutaneous magnetic catheter 300 with respect to the adjustable magnetically driven prosthesis 390.

In various embodiments, cardiac catheterization techniques may be used. For example, as illustrated in FIG. 3, the percutaneous magnetic catheter 300 may enter the right atrium 382 of the heart 380 via the inferior vena cava 386. The percutaneous magnetic catheter 300 may be inserted into a vein in a patient's leg or arm.

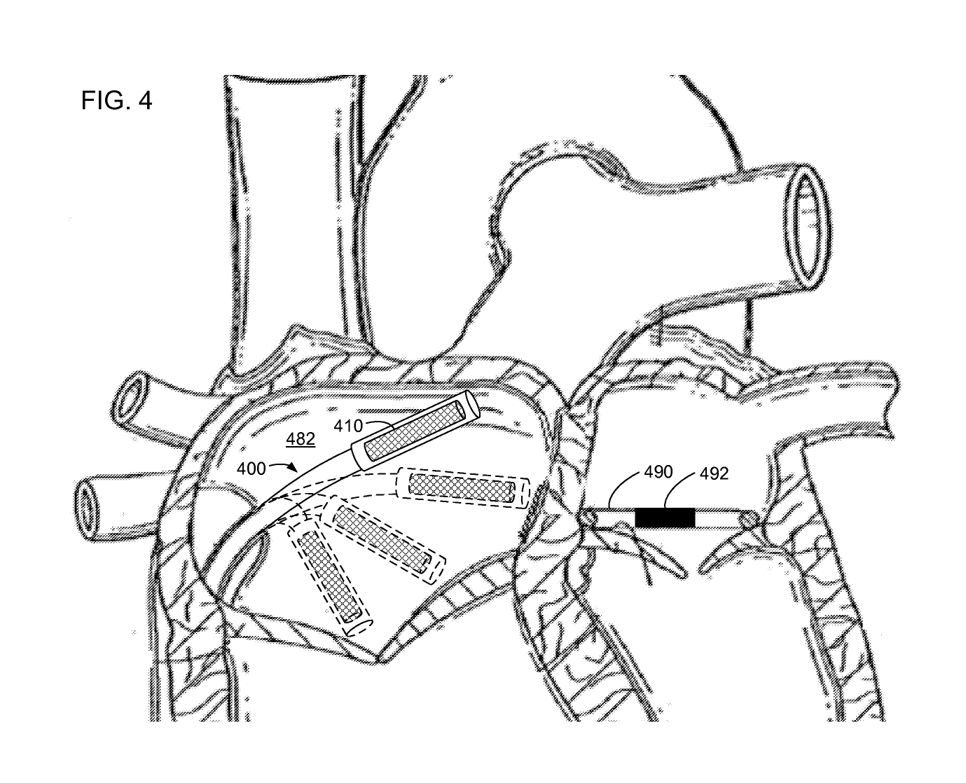

As illustrated in FIG. 4, a percutaneous magnetic catheter 400 may be deflectable in multiple planes according to one embodiment. The percutaneous magnetic catheter 400 may include a deflection mechanism that is configured to cause deflection of a distal end of the percutaneous magnetic catheter 400 based upon motion of the deflection mechanism. Various deflection mechanisms and steerable catheters may be utilized in conjunction with the systems and methods disclosed herein, including those disclosed in U.S. Pat. No. 4,723,936, which is hereby incorporated by reference herein in its entirety.

Various positions of the percutaneous magnetic catheter 400 are shown in FIG. 4 in phantom lines. The ability to deflect the percutaneous magnetic catheter 400 in multiple planes may facilitate the alignment of a magnetic component 410 with respect to an adjustable magnetically driven prosthesis 490. Also, the ability to deflect the percutaneous magnetic catheter 400 in multiple planes may provide greater control to a practitioner, and may thus help to avoid injury to the right atrium 482 during the adjustment procedure. According to one embodiment, the percutaneous magnetic catheter 400 may be deflectable between approximately 0° and 180°. According to other embodiments, the percutaneous magnetic catheter 400 may be deflectable by more than 180°.

In the embodiment illustrated in FIG. 4, the adjustable magnetically driven prosthesis 490 includes a magnetically driven component 492. The magnetically driven component 492 may be in various positions, and accordingly the ability to deflect a distal end of the percutaneous magnetic catheter 400 may allow a practitioner to position the percutaneous magnetic catheter 400 to optimize magnetic coupling with the magnetically driven component 492.

As is described in greater detail below, various embodiments may include a plurality of magnetic components (not shown). In one particular embodiment, a plurality of magnetic components comprising Halbach cylinders are positioned in the right atrium 482. Embodiments comprising a plurality of Halbach cylinders may provide a variety of features and advantages. First, such embodiments may provide greater force to act upon adjustable magnetically driven prosthesis 490. Second, such embodiments may allow for a tighter deflectable radius of curvature of the percutaneous magnetic catheter 400 because of a smaller catheter outer diameter. Third, such embodiments may allow a magnetically driven component 492 in the adjustable magnetically driven prosthesis 390 to be driven in multiple directions by arranging the plurality of magnetic components with respect to the magnetically driven component 492. Fourth, in special cases involving challenging anatomy of the mitral valve, left atrium, or right atrium, such embodiments may allow the plurality of magnetic components to be positioned so that the magnetically driven component 492 may be activated using the combined magnetic field. Artisans will recognize other features and advantages from the disclosure herein.

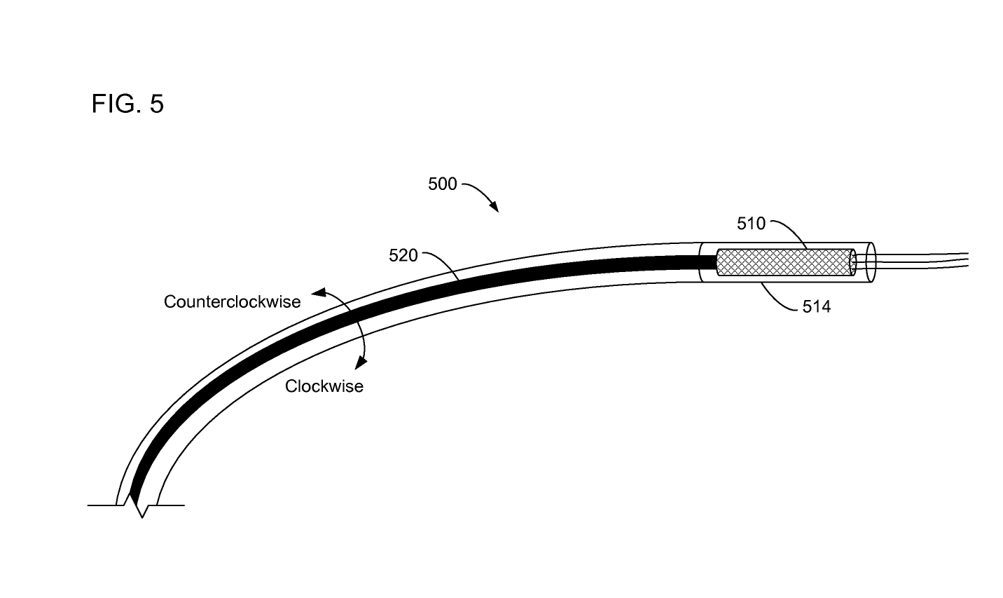

FIG. 5 illustrates one embodiment of a percutaneous magnetic catheter 500 including a protective jacket 514 that surrounds a magnetic component 510. In various embodiments, the protective jacket 514 may be pressed against the right atrium wall without damage to the surrounding tissue. In one embodiment, the protective jacket 514 comprises a polymeric material, such as Polysulfone tubing, which may maintain a separation between the magnetic component 510 and the surrounding tissue. As is further illustrated in FIG. 5, a flexible driveshaft 520 may cause the magnetic component 510 to rotate in either a clockwise or counterclockwise direction.

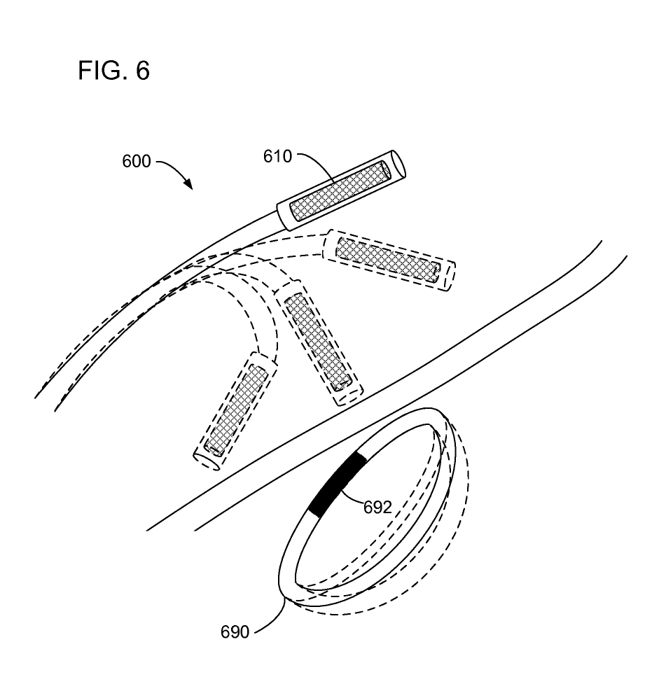

As illustrated in FIG. 6, in various embodiments, a percutaneous magnetic catheter 600 may be manipulated in three dimensions to provide anatomically advantageous positioning of a magnetic component 610 to maximize coupling between the magnetic component 610 and a magnetically driven component 692 of an adjustable magnetically driven prosthesis 690. Various positions of the percutaneous magnetic catheter 600 are shown in FIG. 6 in phantom lines. Also shown in phantom lines are various positions of the adjustable magnetically driven prosthesis 690. As discussed above, the adjustable magnetically driven prosthesis 690 may be adjustable between the illustrated positions utilizing the percutaneous magnetic catheter 600.

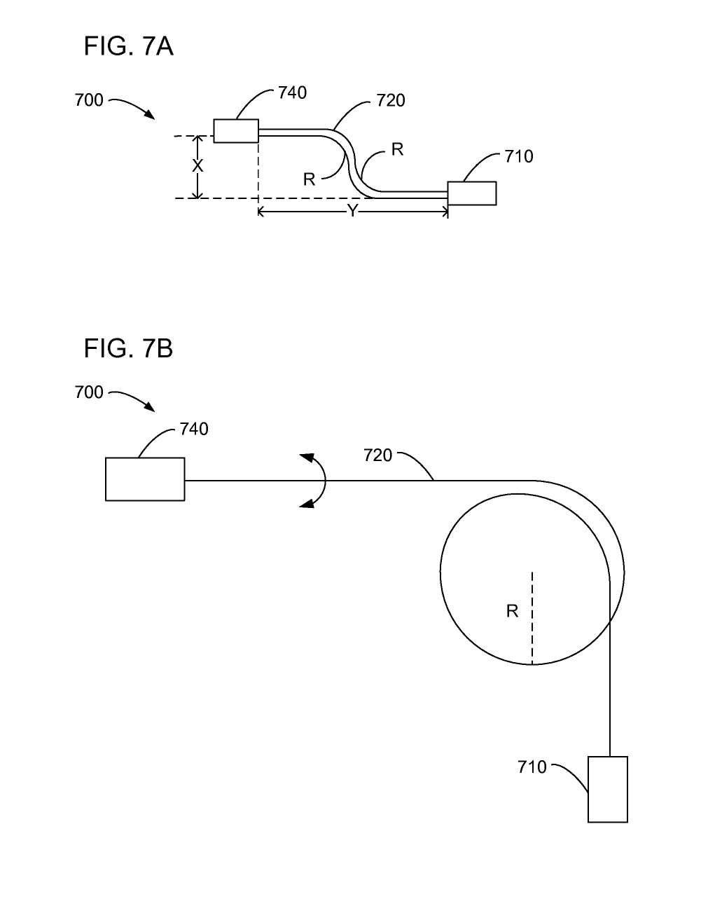

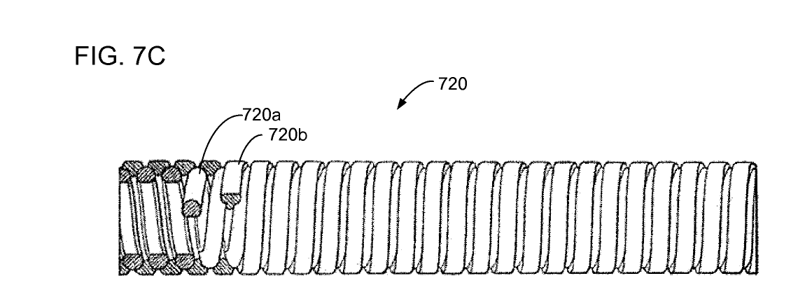

FIGS. 7A and 7B illustrate various attributes of a flexible driveshaft 720, which may be utilized in connection with certain embodiments disclosed herein. The flexible driveshaft 720 allows rotation in both clockwise and counterclockwise directions. The operating environment of a percutaneous magnetic catheter 700 (i.e., driving an adjustable magnetically driven prosthesis in the mitral valve), may be very challenging. Accordingly, the percutaneous magnetic catheter 700 may be purposely over-designed to provide at least two inch-pounds (in-lbs) of torque at the minimum bend radius in order to accommodate years of calcification, overgrowth, and progressing disease stages of a patient with an implanted magnetically driven prosthesis.

In one embodiment, the flexible driveshaft 720 has a minimum bend radius that is calculated using the following formula.

$${R}_{( minimum )} = \frac{{x}^{2} + {y}^{2}}{4 x}$$

FIG. 7A illustrates the variables used in the equation above. The variable x may be referred to as a vertical offset, and the variable y may be referred to as a horizontal offset. In the configuration illustrated in FIG. 7B (i.e., the flexible drive shaft 720 is rotated 360°), the flexible driveshaft 720 may be configured in various embodiments to transfer at least two in-lbs of force to a magnetic component 710 in both clockwise and counterclockwise directions.

In various embodiments, a drive component 740 may include a step motor, which may provide variable ramp-up speed and variable speed in order to provide more torque, according to the following formula. In one embodiment, the drive component 740 may be embodied as Model M42SP-5, available from Mitsumi Electric Co., Ltd., Tokyo, Japan, which has 78.4 mN-m series holding torque and operational torque of 27.6 mN-m while running at 200 pps (12Vdc).

$$Torque = \frac{H . P . \times \text{63,000}}{R . P . M .}$$

In one embodiment, the flexible driveshaft 720 provides at least two in-lbs of torque. Other embodiments may provide as much as 300% more torque as a safety feature. The flexible driveshaft 720 may be designed to be larger in embodiments providing more torque. Similarly, the size or intensity of the magnetic component 710 can be increased in order to provide a stronger magnetic field, and thus greater magnetic coupling with an adjustable magnetically driven prosthesis.

FIG. 7C illustrates a partial cross sectional view of one embodiment of the flexible driveshaft 720. The flexible driveshaft 720 includes an inner core 720a, which is efficient in transferring counterclockwise motion, and an outer core 720b that is efficient in transferring clockwise motion. In one configuration, a flexible drive shaft may be obtained from S.S. White Technologies, Inc., Piscataway, N.J.

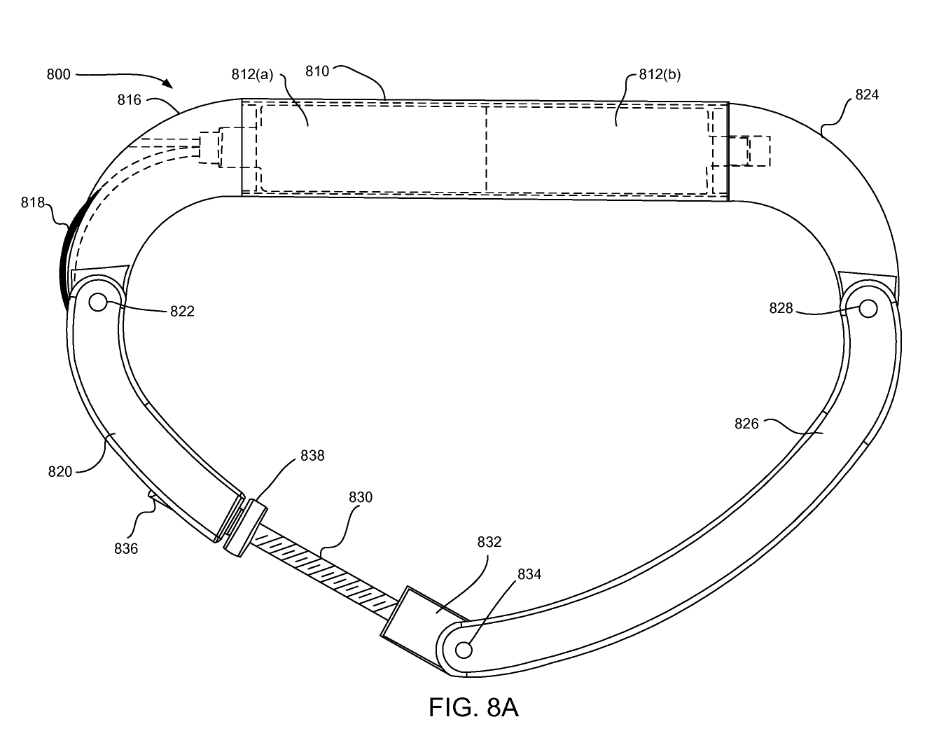

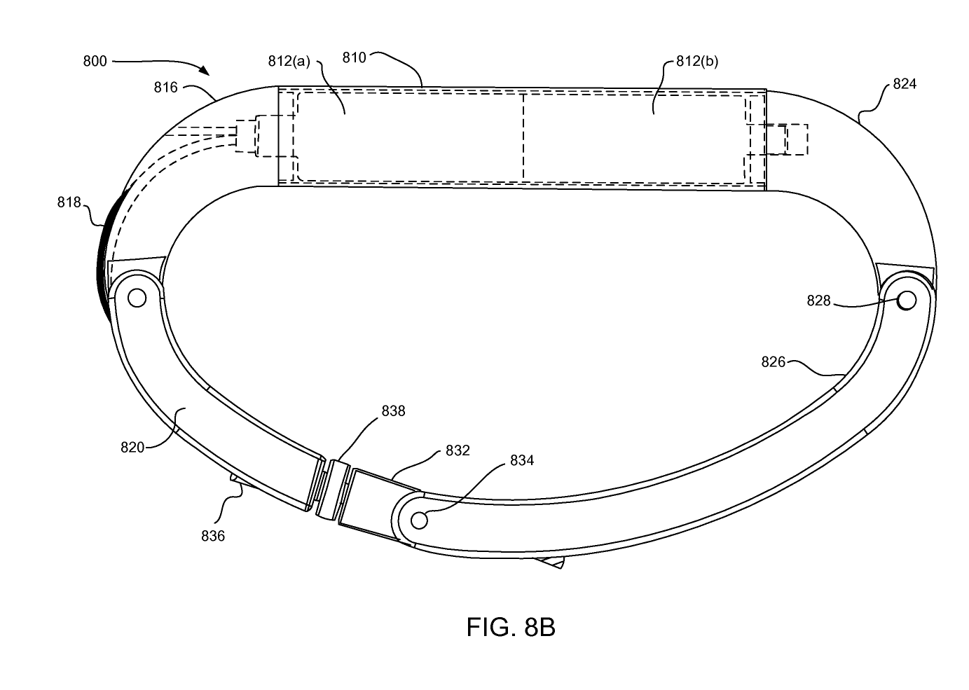

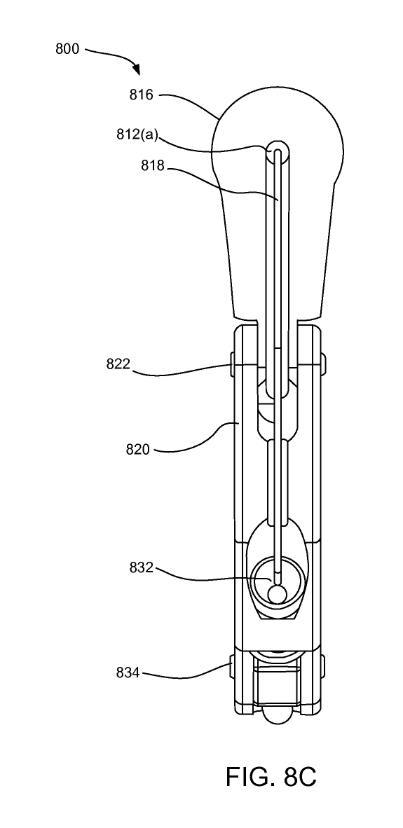

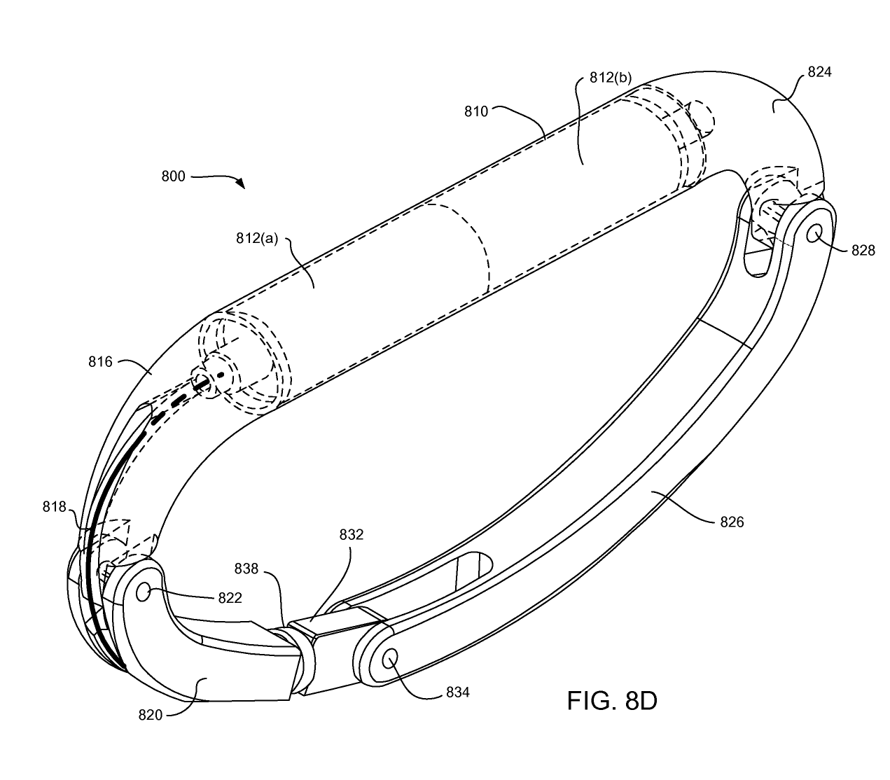

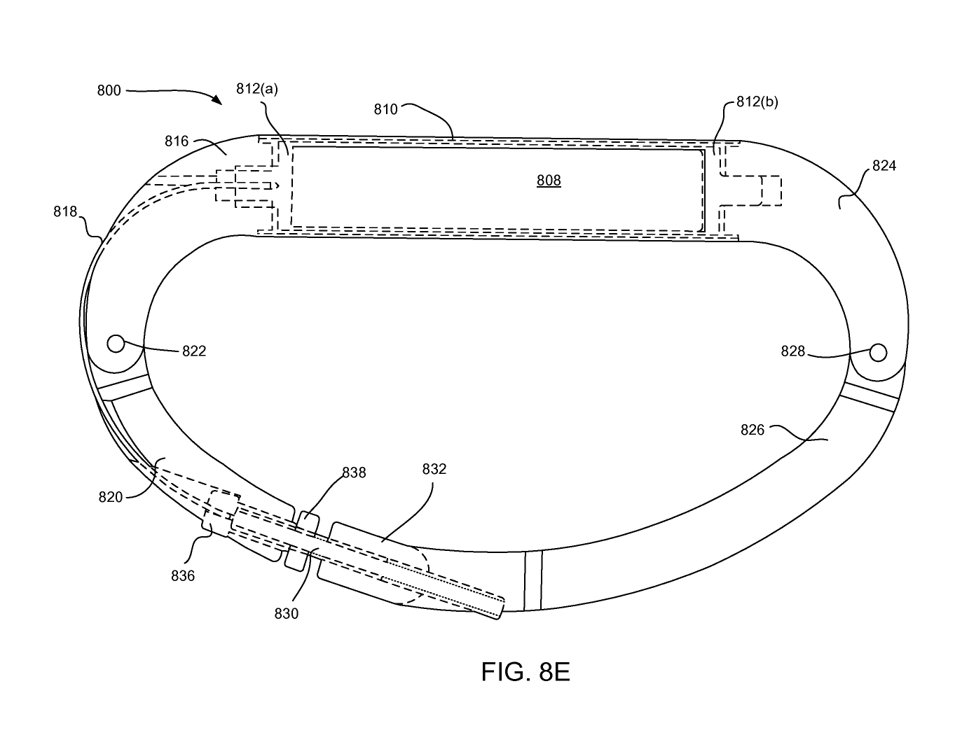

FIGS. 8A, 8B, 8C, 8D, and 8E schematically illustrate an adjustable magnetically driven prosthesis 800 according to one embodiment. The magnetically driven prosthesis in this example is a reversibly adjustable annuloplasty ring. FIG. 8A is a partially transparent top view of the adjustable magnetically driven prosthesis 800 in an anterior/posterior (“AP”) direction extended or plus position. FIG. 8B is a partially transparent top view of the adjustable magnetically driven prosthesis 800 in an AP retracted or minus position. FIG. 8C schematically illustrates a side view of the adjustable magnetically driven prosthesis 800. FIG. 8D is a partially transparent perspective view of the adjustable magnetically driven prosthesis 800. FIG. 8E is another partially transparent top view of the adjustable magnetically driven prosthesis 800.

The adjustable magnetically driven prosthesis 800 includes a body tube 810 for enclosing a magnet housing 812 (including a first end 812(a) and a second end 812(b)) that encases a magnet 808 (FIG. 8E). A first end of the body tube 810 is connected to a first fixed arm 816 and a first end of the magnet housing 812(a) crimps to a first end of a drive cable 818. The first fixed arm 816 is connected to a first swivel arm 820 at a first pin joint 822 (e.g., pivot point). A second end of the body tube 810 is connected to a second fixed arm 824 that is connected to a second swivel arm 826 at a second pin joint 828. The adjustable magnetically driven prosthesis 800 also includes a screw 830 having a first end threaded into a drive nut 832 that is connected to the second swivel arm 826 at a third pin joint 834. A second end of the screw 830 is connected to a drive spindle 836 that is connected to a second end of the drive cable 818. A spindle nut 838 is threaded onto the lead screw 830. The spindle nut 838 retains the drive spindle 836 into the first swivel arm 820.

The magnet housing 812 is engaged with the first fixed arm 816 and the second fixed arm 824 such that rotating the magnet 808 (e.g., using a percutaneous magnetic catheter as disclosed herein) causes the magnet housing 812 to rotate. The rotating magnet housing 812 turns the drive cable 818, which turns the drive spindle 836. The drive spindle 836 rotates the lead screw 830 such that it screws into or out of the drive nut 832. As the lead screw 830 screws into or out of the drive nut 832, the swivel arms 820, 826 pivot at their respective pin joints 822, 828, 834 to reduce or enlarge the size of the ring opening in the AP dimension. Additional embodiments, any of which may be utilized in connection with the systems and methods disclosed herein, are disclosed in U.S. Patent Application Publication No. 2009/0248148, which is hereby incorporated by reference herein for all purposes.

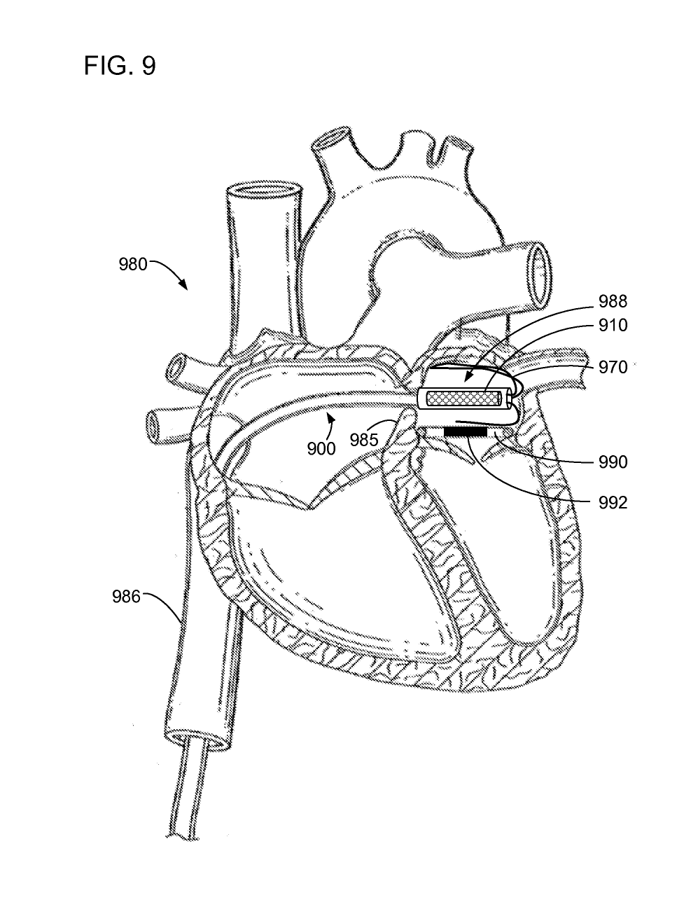

FIG. 9 illustrates a transeptal approach for adjusting a magnetically driven prosthesis 990 using a percutaneous magnetic catheter 900. As illustrated in FIG. 9, the percutaneous magnetic catheter 900 has penetrated the septal wall 985 and is positioned in the left atrium 988 of a heart 980, in order to be nearer to a magnetically driven component 992 of the adjustable magnetically driven prosthesis 990. A transeptal approach may be employed in a variety of circumstances, such as where the magnetic component 910 failed to drive the magnetically driven component 992 because of distance, anatomical abnormality, or a need for a stronger magnetic field. As described above, the percutaneous magnetic catheter 900 may enter the heart 980 via the inferior vena cava 986. Once positioned in left atrium 988, guide wires 970 may be utilized to hold the magnetic component 910 in place during an adjustment procedure.

In certain embodiments, the percutaneous magnetic catheter 900 may be brought into contact with the adjustable magnetically driven prosthesis 990. Such embodiments may allow for the greatest magnetic coupling between the magnetic component 910 and the magnetically driven component 992 because the strength of the magnetic field is a function of distance. Accordingly, by bringing the magnetic component 910 into contact with the magnetically driven component 992, the distance between these components is minimized and the resulting magnetic coupling is maximized.

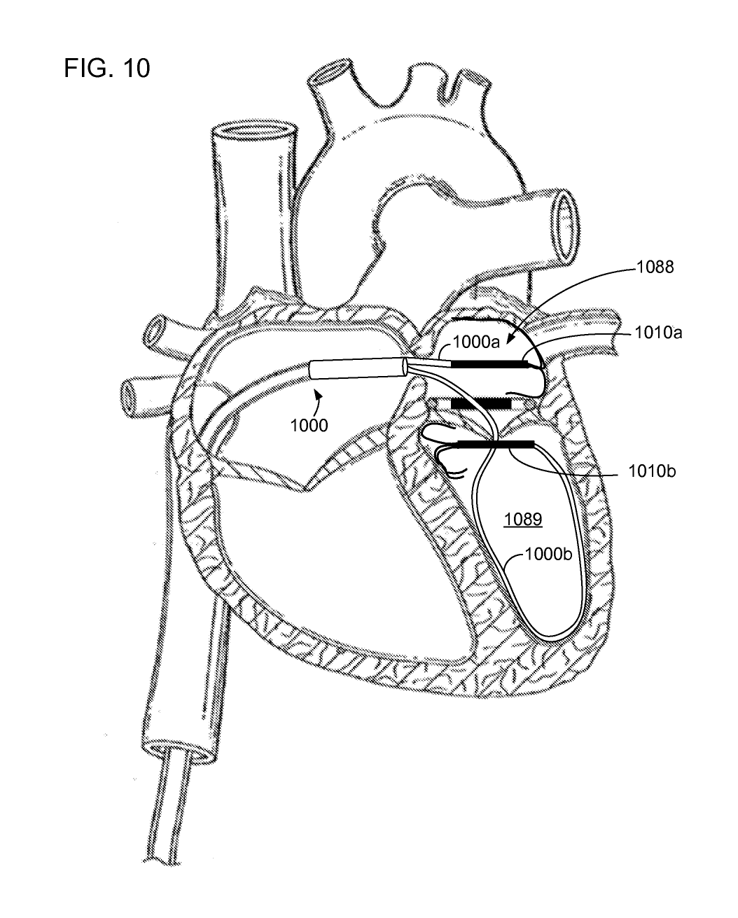

FIG. 10 illustrates one embodiment of a percutaneous magnetic catheter 1000 that includes two magnetic components 1010a and 1010b. The magnetic component 1010a is deployed in the left atrium 1088 and may be connected to an independently manipulable catheter 1000a, while the second magnetic component 1010b is deployed in the left ventricle 1089 and may be connected to another independently manipulable catheter 1000b. In the illustrated embodiment, the independently manipulable catheters 1000a and 1000b are each connected to the percutaneous magnetic catheter 1000. In other embodiments, separate percutaneous magnetic catheters may be utilized for each magnetic component 1010a, 1010b. In one embodiment, an inflatable balloon (not shown) may be used when deploying the independently manipulable catheters 1000a, 1000b to prevent the respective magnetic components 1010a, 1010b from physically connecting to one another due to magnetic coupling.

In various embodiments including a single or a plurality of magnetic components, a variety of imaging techniques may also be utilized to assist in the positioning of each magnetic component. For example, X-ray or ultrasound imaging techniques may be utilized.

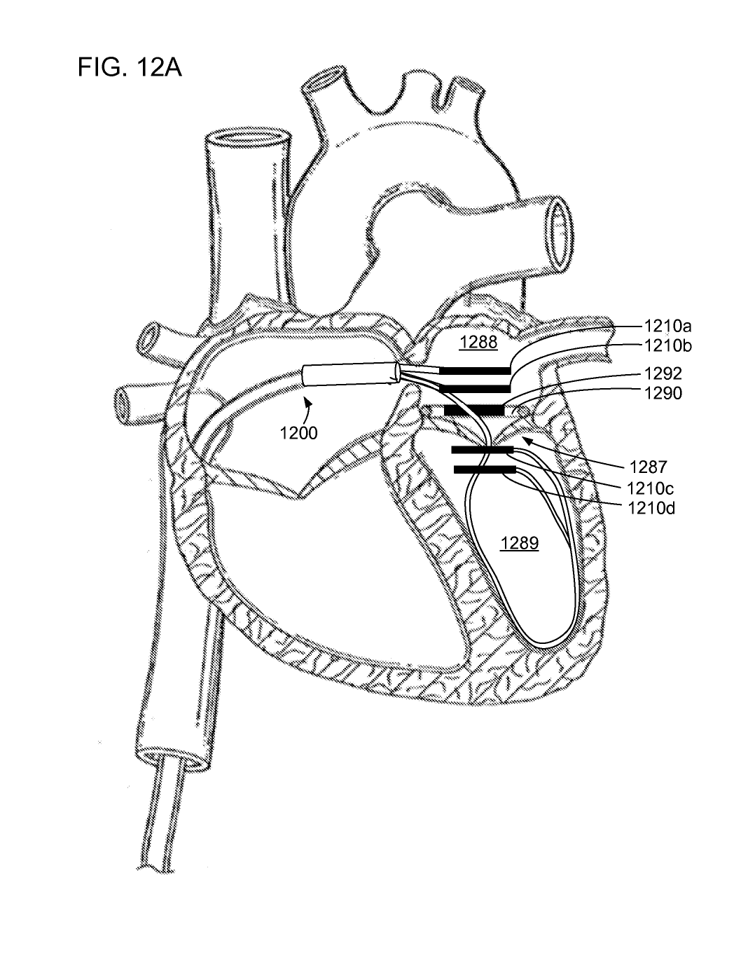

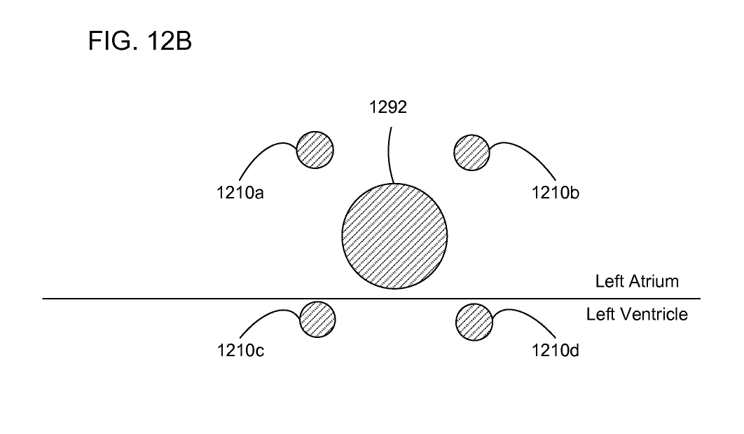

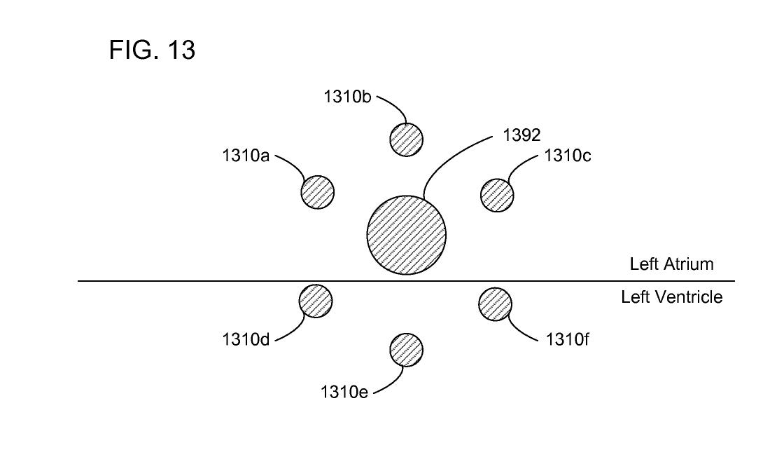

A variety of embodiments are contemplated that include a plurality of magnetic components. Any number of magnetic components may be utilized in order to achieve a desired magnetic field strength. The embodiment illustrated in FIG. 10 includes two magnetic components. An embodiment illustrated in FIGS. 12A and 12B includes four magnetic components, while FIG. 13 illustrates an embodiment that includes six magnetic components.

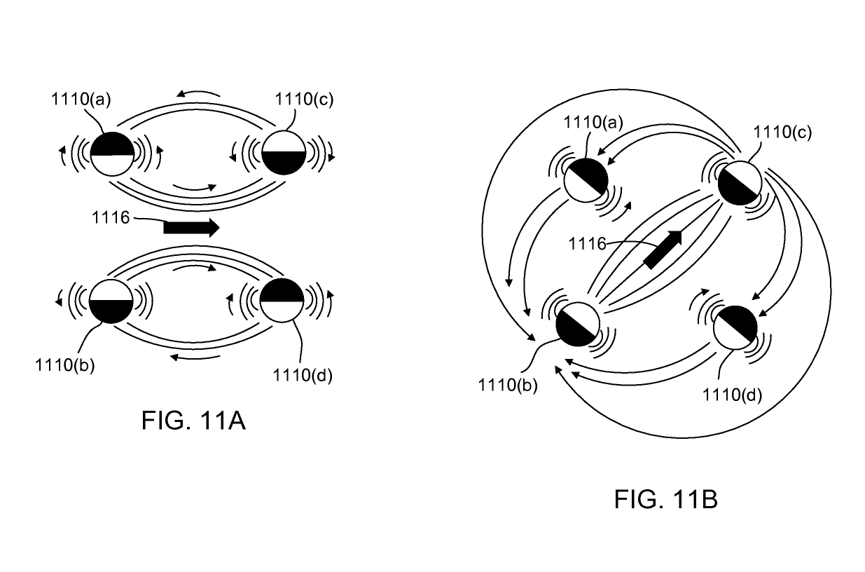

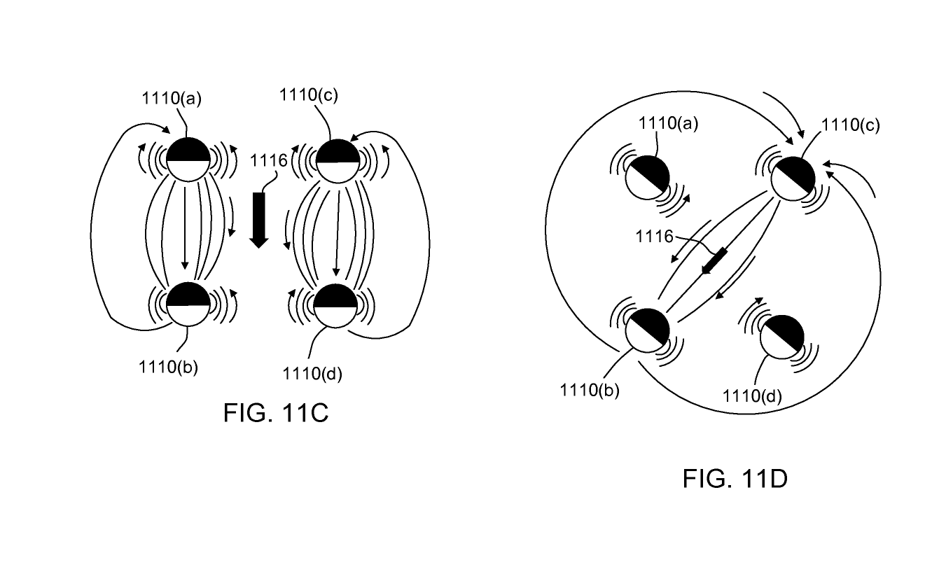

FIGS. 11A, 11B, 11C, and 11D illustrate how the individual magnetic fields of a plurality of magnetic components 1110(a)-1110(d) may be utilized in order to produce a combined magnetic field 1116 capable of adjusting an adjustable magnetically driven prosthesis (not shown). The strength of the combined magnetic field 1116 in the area between the plurality of magnetic components 1110(a)-1110(d) is based on the polar alignment (e.g., north and south poles) of each magnet 1110. For example, FIGS. 11A, 11B, 11C, and 11D schematically illustrate end views of magnets 1110. In the illustrated examples, a first magnetic pole (e.g., north) is represented by a white semicircle and a second magnetic pole (e.g., south) is represented by a black semicircle.

In FIG. 11A, the magnets 1110 are in an anti-aligned (Halbach) arrangement with the line separating the magnetic poles in each magnet 1110 set at a 0° offset from a horizontal direction. In this arrangement, the magnetic fields from each magnet 1110 form a combined magnetic field 1116 in the direction indicated in the central area of the magnet array, while reducing the magnetic field in areas outside of the magnet array. When the magnets 1110 are rotated in unison, the combined magnetic field 1116 in the central area of the magnet array rotates in the opposite direction. For example, FIG. 11B illustrates the magnets 1110 rotated 45° in a clockwise direction as compared to the arrangement of FIG. 11A. Accordingly, the combined magnetic field 1116 is also rotated 45°, but in the counterclockwise direction.

In FIG. 11C, the magnets 1110 are in an aligned arrangement such that like magnetic poles are all facing the same direction (e.g., all north poles face in the same direction). Further, the line separating the magnetic poles in each magnet 1110 is set at a 0° offset from a horizontal direction. This arrangement results in a combined magnetic field 1116 in the orientation indicated in the central area of the magnet array. However, in some orientations, the combined magnetic field generated in the central region by the aligned arrangement shown in FIG. 11C may not be as great as that of the Halbach arrangement shown in FIG. 11A. Further, the combined magnetic field in the central region of the magnet array may decrease as the magnets 1110 are rotated. For example, FIG. 11D illustrates the magnets 1110 rotated 45° in a clockwise direction as compared to the arrangement of FIG. 11C. The combined magnetic field 1116 in the central area of the magnet array is also rotated 45° in the clockwise direction. However, the magnitude of the combined magnetic field 1116 in the central region of the magnet array is reduced due to counteracting magnetic fields generated by the magnet 110(a) and the magnet 110(d).

In embodiments including a plurality of magnetic components, an encoder may be associated with each magnetic component to monitor the angular position of each magnetic component. Further, a controller may be utilized to maintain synchronicity between the plurality of magnetic components. In various embodiments, the encoders may be a part of a feedback system that allows for precise control of each magnetic component and the optimization of the resulting combined magnetic field. Accordingly, a decoder may be used for embodiments that include a single magnetic component used to adjust an annuloplasty ring.

FIG. 12A illustrates an embodiment of a percutaneous magnetic catheter 1200 that includes two magnetic components 1210a and 1210b positioned in the left atrium 1288 and two magnetic components 1210c and 1210d positioned in the left ventricle 1289. As illustrated, the magnetic components 1210c and 1210d may pass through an adjustable magnetically driven prosthesis 1290, the mitral valve 1287, and into the left ventricle 1289. The adjustable magnetically driven prosthesis 1290 includes a magnetically driven component 1292, as discussed above.

FIG. 12B schematically illustrates a cross-sectional view of the embodiment illustrated in FIG. 12A. As shown in FIG. 12B, the magnetic components 1210a, 1210b, 1210c, 1210d (magnetic components 1210a-1210d) may be arranged around the magnetically driven component 1292 of an adjustable magnetically driven prosthesis (not shown). In certain embodiments, the magnetic components 1210a-1210d may include permanent magnets, while in other embodiments the magnetic components 1210a-1210d may include electromagnets. The rotation of the magnetic components 1210a-1210d, in the case of permanent magnets, and the activation of the magnetic components 1210a-1210d, in the case of electromagnets, may be controlled using any appropriate control mechanism.

In the embodiment illustrated in FIG. 13, three magnetic components 1310a, 1310b, 1310c are positioned in the left atrium of a heart, and three magnetic components 1310d, 1310e, 1310f are positioned in the left ventricle of the heart. The embodiment illustrated in FIG. 13 may be utilized in cases requiring a stronger magnetic field to rotate a magnetically driven component 1392 of an adjustable magnetically driven prosthesis (not shown).

For different anatomical positions of the mitral valve where the orientation of the driven magnetic component is more to the septal-lateral direction, the drive magnetic component catheters can be arranged in the right atrium, left atrium, and left ventricle to optimize the magnetic force applied to an adjustable magnetically driven prosthesis. Guide wires used to brace the magnetic components within the respective chambers are not shown in FIGS. 12A, 12B, and 13, but may be utilized in any of these embodiments.

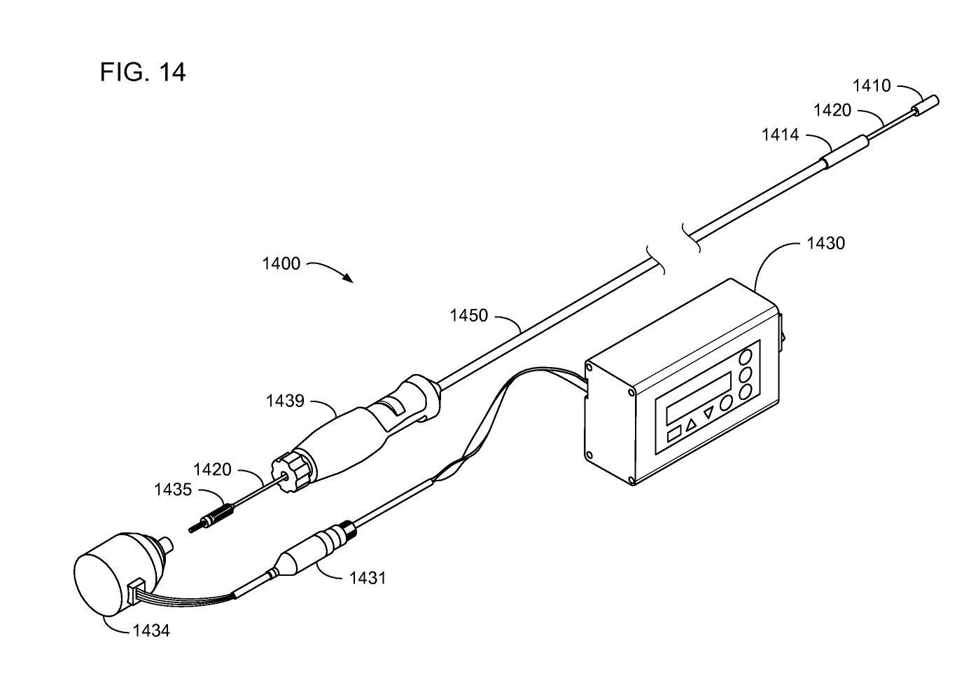

FIG. 14 illustrates one embodiment of a magnetic catheter system 1400. The magnetic catheter system 1400 includes a step motor controller 1430. The step motor controller 1430 may allow for control of a magnetic component 1410. In various embodiments, the magnetic component 1410 may be embodied as a Halbach cylinder, as another form of permanent magnet, or as an electromagnet. The step motor controller 1430 may control the direction of rotation and the speed of rotation of the magnetic component 1410. In embodiments in which the magnetic component 1410 comprises an electromagnet, the step motor controller 1430 or another controller may selectively activate the electromagnet.

The step motor controller 1430 may be in electrical communication with a step motor 1434 by way of an electrical connector 1431. The step motor 1434 may be configured to provide clockwise and counterclockwise rotation. A variety of types of motors may embody the step motor 1434.

The step motor 1434 may be configured to couple to a flexible driveshaft 1420. The flexible driveshaft 1420 may include a driveshaft connector 1435 configured to be received within the step motor 1434. The flexible driveshaft 1420 may extend within a catheter 1450 and may be coupled to the magnetic component 1410. A protective jacket 1414 may be disposed at the distal end of the catheter 1450. The protective jacket 1414 may be configured to receive the magnetic component 1410. The magnetic component 1410 may be extended from the protective jacket 1414, as illustrated in FIG. 14, during an adjustment procedure.

A catheter grip 1439 may be used to position the magnetic component 1410 in a desired location during an adjustment procedure. In one embodiment, the magnetic component 1410 may be designed to magnetically couple to a magnetically driven component (not shown) in an adjustable magnetically driven prosthesis (not shown) at a distance of approximately 1.5 inches. In other embodiments, the distance may be greater than or less than 1.5 inches.

Those having skill in the art will recognize that many changes may be made to the details of the above-described embodiments without departing from the underlying principles disclosed herein. The scope of the present invention should, therefore, be determined only by the following claims.

1. A magnetic catheter system, comprising:
a catheter having a proximal end and a distal end;
a deflection mechanism configured to cause deflection of the distal end of the catheter based on motion of the deflection mechanism;
a drive motor configured to selectively generate a rotational force;
a drive motor controller configured to selectively activate the drive motor;
a rotatable magnetic component; and
a flexible driveshaft disposed within the catheter, the flexible driveshaft coupled to the drive motor and rotatable magnetic component and configured to transfer the rotational force generated by the drive motor to the rotatable magnetic component.

2. The system of claim 1, wherein the drive motor comprises a step motor.

3. The system of claim 1, further comprising:
at least one adjustable guide wire configured to be braced against one or more objects and to stabilize the magnetic component while the drive motor generates the rotational force.

4. The system of claim 1, wherein the rotatable magnetic component is movable between an extended position and a retracted position, and wherein the system further comprises:
a protective jacket disposed at the distal end of the catheter, the protective jacket configured to receive the rotatable magnetic component in the retracted position, and wherein the rotatable magnetic component extends at least partially from the protective jacket in the extended position.

5. The system of claim 4, wherein the protective jacket comprises a polymeric material.

6. The system of claim 1, wherein the magnetic component comprises a Halbach cylinder.

7. The system of claim 1, wherein the drive motor is operable to generate a clockwise rotational force and a counterclockwise rotational force.

8. The system of claim 1, wherein the flexible driveshaft comprises an inner core and an outer core.

9. The system of claim 1, wherein the drive motor and a flexible driveshaft transfer at least two in-lbs of torque to the magnetic component.

10. The system of claim 1, wherein the distal end of the catheter is deflectable between approximately 0° and 180°.

11. The system of claim 1, wherein the flexible driveshaft has a minimum bend radius proportional to the sum of the squares of a vertical offset and a horizontal offset divided by a multiple of the vertical offset.

12. The system of claim 1, wherein the distal end of the catheter is configured to penetrate a septal wall.

13. The system of claim 1, further comprising a plurality of rotatable magnetic components.

14. A method of adjusting a magnetically driven prosthesis, comprising:
positioning a distal end of a catheter near a magnetically driven prosthesis, the distal end of the catheter comprising a rotatable magnetic component;
manipulating a deflection mechanism configured to cause deflection of the distal end of the catheter based on motion of the deflection mechanism;
selectively generating a rotational force;
rotating the magnetic component using the rotational force in proximity to the magnetically driven prosthesis such that variations in a magnetic field caused by rotation of the magnetic component adjusts the magnetically driven prosthesis.

15. The method of claim 14, further comprising:
bracing at least one guide wire against one or more objects to stabilize the magnetic component while the magnetic component is rotating.

16. The method of claim 14, further comprising:
extending the magnetic component from a retracted position in which the magnetic component is received within a protective jacket, to an extended position in which the magnetic component extends at least partially from the protective jacket.

17. The method of claim 16, wherein the protective jacket comprises a polymeric material.

18. The method of claim 14, wherein rotating the magnetic component comprises selectively rotating the magnetic component in a clockwise orientation and rotating the magnetic component in a counterclockwise orientation.

19. The method of claim 14, wherein positioning a distal end of a catheter near a magnetically driven prosthesis comprises penetrating a septal wall.

20. The method of claim 14, further comprising:
positioning a plurality of magnetic components in proximity to the magnetically driven prosthesis; and
rotating the plurality of magnetic components such that variations in a magnetic field caused by rotation of the plurality of magnetic components adjusts the magnetically driven prosthesis.

21. The method of claim 20, wherein positioning a plurality of magnetic components in proximity to the magnetically driven prosthesis comprises positioning the plurality of magnetic components in a right atrium in a non-aligned, Halbach configuration.
